(19) United States
(12) Patent Application Publication
(10) Pub. No.: US 2018/0099059 A1
(43) Pub. Date: Apr. 12, 2018

(54) METHODS AND APPARATUS FOR CELL TRACKING AND MOLECULAR IMAGING

(71) Applicants: John A. Hossack, Charlottesville, VA (US); Adam Joseph Dixon, Charlottesville, VA (US); Edwin Lu, Charlottesville, VA (US)

(72) Inventors: John A. Hossack, Charlottesville, VA (US); Adam Joseph Dixon, Charlottesville, VA (US); Edwin Lu, Charlottesville, VA (US)

(21) Appl. No.: 15/729,164

(22) Filed: Oct. 10, 2017

Related U.S. Application Data
(60) Provisional application No. 62/405,364, filed on Oct. 7, 2016.

(51) Int. Cl. A61K 49/22

(52) U.S. Cl. CPC A61K 49/223

(57) ABSTRACT

A composition for cell tracking and molecular imaging containing perfluorocarbon (“PFC”) droplets having a liquid PFC core enclosed within a stabilizing shell and embedded with solid nanoparticles. The solid nanoparticles act as nucleating agents for reducing the activation pressure of the liquid PFC core required to transition the liquid PFC core to a gaseous microbubble thereby permitting the use of more body-temperature stable longer chain PFCs in the liquid PFC core. The improved stability of the PFC droplets with a reduced or limited increase in the activation pressure required due to the nucleating nanoparticles improves the efficacy of using the PFC droplets as phase-change contrast agents.

### CLAIM OF PRIORITY

This patent application claims the benefit of priority, under 35 U.S.C. Section 119(e), to Adam Joseph Dixon et al., U.S. Patent Application Ser. No. 62/405,364, entitled “METHODS AND APPARATUS FOR CELL TRACKING AND MOLECULAR IMAGING,” filed on Oct. 7, 2016 (Attorney Docket No. 1036.288PRV), each of which is hereby incorporated by reference herein in its entirety.

### TECHNICAL FIELD

This document pertains generally, but not by way of limitation, to cell tracking and molecular imaging methods and systems.

### BACKGROUND

Phase-change contrast agents (“PCCA”) can comprise liquid droplets that can be introduced into a patient where the droplets can be internalized by the target cells or accumulate at the target site. High-pressure acoustic waves, such as ultrasound, can be used to excite the droplets until the droplets exceed a pressure threshold and undergo acoustic droplet vaporization (“ADV”) to become gaseous microbubbles. The phase change of each droplet to a microbubble generates an acoustic signal detectable by ultrasonic imaging transducers to localize the resulting microbubble, and correspondingly the target cells or site, within the patient's body in the time and space.

Liquid droplets comprising perfluorocarbon (“PFC”) are attractive options for PCCAs as PFC droplets are inert and non-toxic in low dosages. However, shorter carbon length PFCs (e.g., decafluorobutane) are unsuitable for PCCAs as droplets comprising shorter carbon length PFCs are less stable at body temperatures (37° C.) and prone to spontaneous phase changes at body temperatures. PCCA stability can be an important characteristic of PCCAs as there can be a significant delay between the introduction of the PCCA and the application of ultrasonic energy for imaging. While longer carbon length PFCs (e.g., dodecafluoropentane) can provide a more stable droplet at body temperatures, longer carbon length PFCs can have a higher pressure threshold for transition from liquid to gas. The greater ultrasonic energies required to exceed the higher pressure thresholds for longer carbon length PFC droplets can exceed the Food and Drug Administration limits on ultrasound exposure (Mechanical Index>1.9) thereby limiting the usability of PFC droplets as PCCAs.

The inherent tradeoff between stability and safely causing the phase change of the liquid droplets to microbubbles discourages the use of PFC droplets as PCCAs.

### OVERVIEW

The present inventors have recognized, among other things, that a problem to be solved can include the drawbacks of PFC droplets as PCCAs due to the elevated activation pressures required for body-temperature stable PFC droplets. In an example, the present subject matter can provide a solution to this problem, such as by providing PFC droplets having a liquid PFC core enclosed within a stabilizing shell and embedded with solid nanoparticles. The solid nanoparticles can act as nucleating agents that reduce the activation pressure of the liquid PFC core required to transition the liquid PFC core to a gaseous microbubble thereby permitting the use of more body-temperature stable longer chain PFCs in the liquid PFC core. The improved stability of the PFC droplets with a reduced or limited increase in the activation pressure required due to the nucleating nanoparticles improves the efficacy of using the PFC droplets as PCCAs.

This overview is intended to provide an overview of subject matter of the present patent application. It is not intended to provide an exclusive or exhaustive explanation of the present subject matter. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings generally illustrate, by way of example, but not by way of limitation, various embodiments discussed in the present document.

FIG. 1 is a schematic diagram illustrating of a PFC droplet according to an example of the present disclosure.

FIG. 2 is a schematic diagram illustrating a method of forming a PFC droplet according to an example of the present disclosure.

FIG. 3A is a transmission electron microscope image of an MSN cluster before embedding within a liquid PFC core according to an example of the present disclosure.

FIG. 3B is a transmission electron microscope image of a PFC droplet having a liquid PFC core without embedded F-MSNs according to an example of the present disclosure.

FIG. 3C is a transmission electron microscope image of a PFC droplet having a liquid PFC core having embedded F-MSNs according to an example of the present disclosure.

FIG. 4 is a schematic diagram of a microfluidic device for forming a PFC droplet according to an example of the present disclosure.

FIG. 5 is a schematic diagram illustrating a method of administering PFC droplets to a patient through ex vivo ingestion of PFC droplets into donor cells according to an example of the present disclosure.

FIG. 6 is a schematic diagram illustrating a method of administering PFC droplets to a patient through direct injection into a patient according to an example of the present disclosure.

FIGS. 7A-7E are transmission electron microscope of a PFC droplet being converted to a gaseous microbubble over about 500 ns according to an example of the present disclosure.

FIG. 8 is a basic, schematic representation of an ultrasound system according to an aspect of an embodiment of the present invention.

FIG. 9 is a block diagram illustrating an example of a machine upon which one or more aspects of embodiments of the present invention can be implemented.

### DETAILED DESCRIPTION

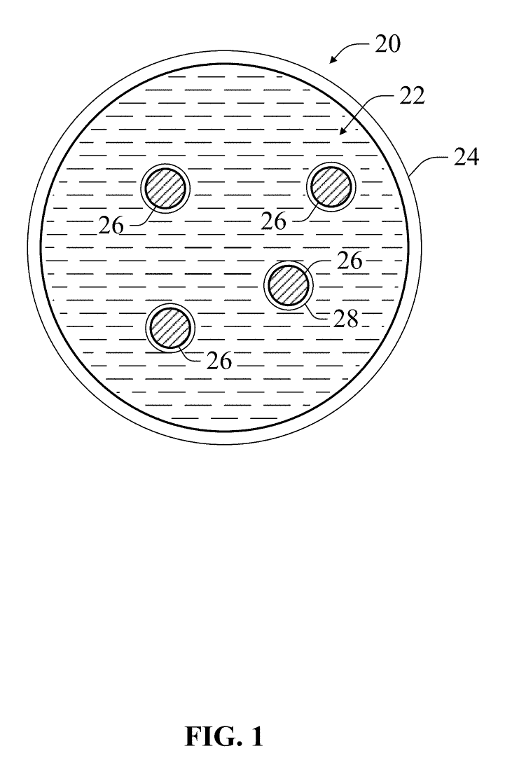

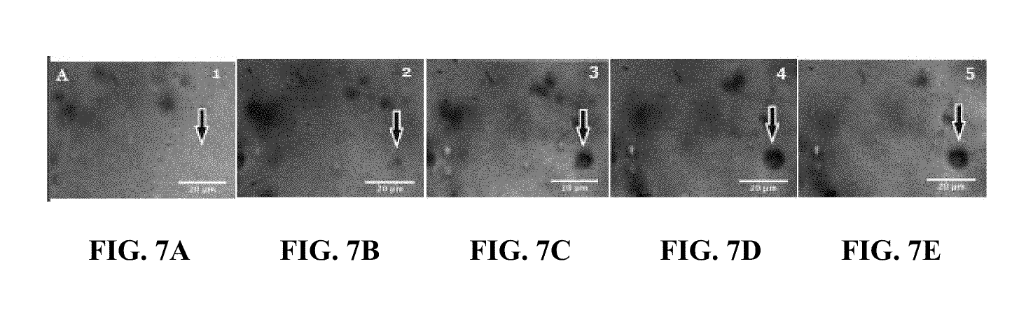

As illustrated in FIG. 1, a PFC droplet 20 for cell tracking and molecular imaging, according to an example of the present disclosure, can comprise a liquid PFC core 22 enclosed within a stabilizing shell 24 and having solid nanoparticles 26 embedded therein. Energy can be applied to the PFC droplet 20 until the internal pressure within the liquid PFC core 22 exceeds a predetermined threshold and undergoes a phase change thereby transitioning the PFC droplet 20 to a microbubble as illustrated in FIGS. 7A-E. The phase change of the PFC droplet 20 to a gaseous microbubble can create an auditory signal, an optical signal, or both detectable to determine the location of the microbubble in time and space. The solid nanoparticles 26 can act as nucleating agents that encourages cavitation of the liquid PFC core 22 thereby reducing the activation pressure for the phase change. The reduced activation pressure allows more stable PFCs in the PFC core 22 that would otherwise require prohibitively high activation pressures.

In an example, the PFC core 22 can comprise PFC liquids including, but not limited to PFC₃, PFC₄, PFC₅, and PFC₆. The PFC core 22 can comprise a single PFC liquid type as the combination of PFC types reduces the pressure required to initiate cavitation at the expense of thermal stability.

In an example, the stabilizing shell 24 can comprise a biocompatible lipid shell suitable for the formation of microbubble formations. In at least one example, the lipid shell can comprise a 9:1 mole ratio of 1,2-Distearoyl-sn-glycero-3-phosphocholine (“DSPC”) and 1,2-dis-tearoyl-sn-glycero-3-phosphoethanolamine-N-[amino(polyethylene glycol)-2000 (“DSPE-PEG2000”). The stabilizing shell 24 can comprise a payload including, but not limited to optical contrast agents, pharmaceuticals, antigens, genetic material, and combinations thereof. In at least one example, the PFC droplet 20 can comprise a plurality of shell layers.

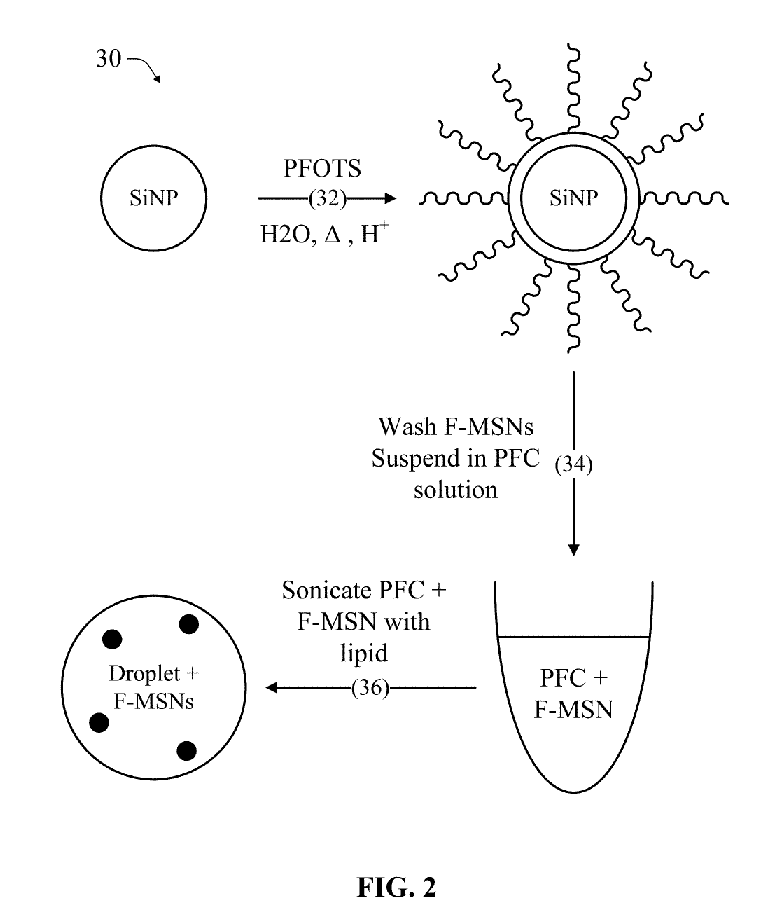

In an example, the nanoparticles 26 can act as a heterogeneous nucleating agent promoting the transition of the liquid PFC core 22 to a gaseous microbubble. The nanoparticles 26 can be roughened to increase the overall surface area of the nanoparticles 26 and to form nanocavities on the outer surface of the nanoparticles 26. The nanocavities can act as nucleation sites at which a bubble can be nucleated thereby reducing the overall energy required to cavitate the liquid PFC core 22. The nanoparticles 26 can comprise silica nanoparticles (“SiNP”) to provide tunable particle diameter, porosity, and cavity size. In at least one example, the nanoparticles 26 can comprise mesoporous silica nanoparticles (“MSNs”). In at least one example, the nanoparticles 26 can also comprise gold or iron oxide nanoparticles 26. For ease of description, the nanoparticles 26 are described as MSNs. The MSNs can comprise SiNP, gold nanoparticles, iron oxide nanoparticles, or combinations thereof. As illustrated in FIG. 2, the nanoparticles 26 can be treated with a fluorinating or hydrophobic surface 28 to permit solvation of the nanoparticles within the PFC core. At high peak-negative pressures, dissolved gases within the PFC core 22 can preferentially leave the liquid phase and form a bubble on the hydrophobic surface 28 of the nanoparticle thereby reducing the energy required to cavitate the liquid PFC phase.

### Preparation of PFC Droplets

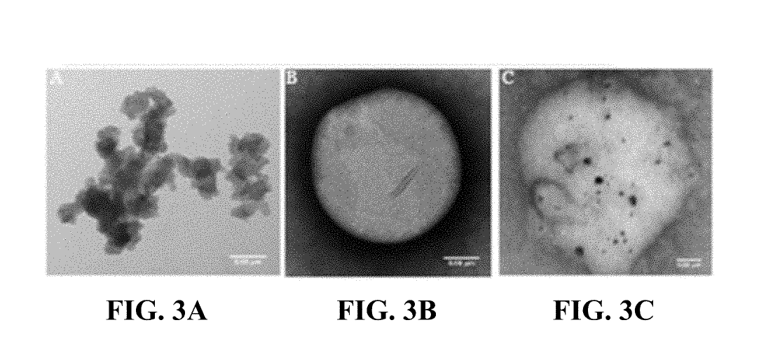

As illustrated in FIG. 2 and FIGS. 3A-C, a PFC droplet 20, according to an example of the present disclosure, can be formed by a method 30 comprising a preparation of nanoparticles 26 step 32, immersion of the nanoparticles 26 in an aqueous PFC solution step 34, and a PFC droplet 20 formation step 36.

As shown in FIG. 3A, in an example, the nanoparticles 26 preparation step 32 can comprise providing nanoparticles 26 having a predetermined roughness and nanocavity depth. In the illustrated example, the nanoparticles 26 can comprise SiNP, and specifically MSNs, having nanocavities between about 1 to about 50 nm in radius. The preparation step 32 can also comprise forming a fluorinated or hydrophobic surface coating 28 on the outer surfaces of the nanoparticles 26 to promote solubility of the nanoparticles 26 in the liquid PFC core 22. A fluorine surface coating can be formed by reacting the MSNs with 1H,1H,2H,2H-Perfluorooctyltriethoxysilane (“PFOTS”) to form fluorinated-MSNs (“F-MSNs”). A hydrophobic surface coating can be formed by reacting the MSNs with hexadecyltrimethoxysilane (“C₁₆TMS”) to form hydrophobic-MSNs (“H-MSNs”).

In an example, the immersion step 34 can comprise combining the F-MSNs with an aqueous PFC solution that comprises a PFC and lipids for the formation of the stabilizing shell. As discussed above, the MSNs can be treated to form a fluorinated surface coating 28 (F-MSNs) or a hydrophobic surface coating 28 (H-MSNs). For ease of description, the functionalized nanoparticles 26 are described as F-MSNs, but can reference F-MSNs, H-MSNs, or combinations thereof. The aqueous PFC solution can comprise a volumetric ratio of PEC to other solution components of about 1:9. The PEC aqueous solution can comprise about 2 mg/ml of a lipid mixture comprising a 9:1 mole ratio of DSPC:DSPE-PEG2000. In an example, before immersion in the aqueous PFC solution, the F-MSNs can be centrifugally washed at least two times in an aqueous ethanol solution and at least two times with a PEC solution comprising PFC₆ or other suitable PFCs.

As shown in FIG. 3C, in an example, the PFC droplet 20 formation step 36 can comprise sonicating the aqueous PFC solution containing the F-MSNs to form the PFC within the aqueous PFC solution into liquid PFC cores 22 enclosed within a stabilizing shell 24 and having solid nanoparticles 26 embedded therein. As shown in FIG. 3B, in at least one example, this method can result in certain PFC droplets 20 can comprise a liquid PFC core 22 without embedded solid nanoparticles 26 depending on the dispersion of the nanoparticles 26 within the aqueous PFC solution. The PFC droplets 20 can be centrifugally washed with a saline solution to remove excess lipids and preserve only the PFC droplets 20. The PFC droplets 20 can be filtered with membrane filters to only select droplets below a predetermined size threshold, such as about 200 nm.

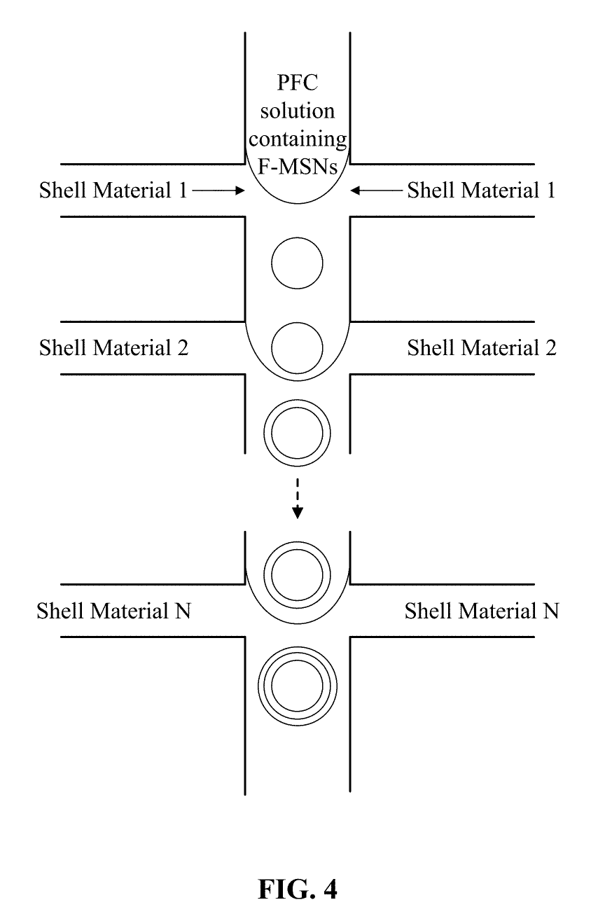

As illustrated in FIG. 4, a PFC droplet 20, according to an example of the present disclosure, can be formed in a microfluidic device 40 having a central channel 42 and a plurality of secondary channels 44 intersecting the central channel 42. The central channel 42 and secondary channels 44 can have sub-millimeter dimensions. In at least one example, the microfluidic device 40 can comprise a substrate of PDMS, glass, or other substrate material that can be etched to form the central channel 42 and secondary channels 44.

In operation, an aqueous PFC solution containing dispersed F-MSNs is introduced into one end of the central channel 42 and fed toward the junction of the central channel 42 and the first secondary channel 44. A shell material for the first shell layer is introduced through the first secondary channel 44 to form a first shell layer on the PFC solution as the PFC solution moves the corresponding intersection 46 between the central channel 42 and the first secondary channel 44. As illustrated in FIG. 3, the steps can be repeated to apply as many shell layers on the liquid PFC core as desired. The microfluidic approach for forming the PFC droplets 20 can be used to precisely size the PFC droplets 20 and produce uniformly sized PFC droplets 20. In an example, the microfluidic approach can be used to form the PFC droplets 20 with a minimum size of about 5 μm.

### Administration of the PFC Droplets

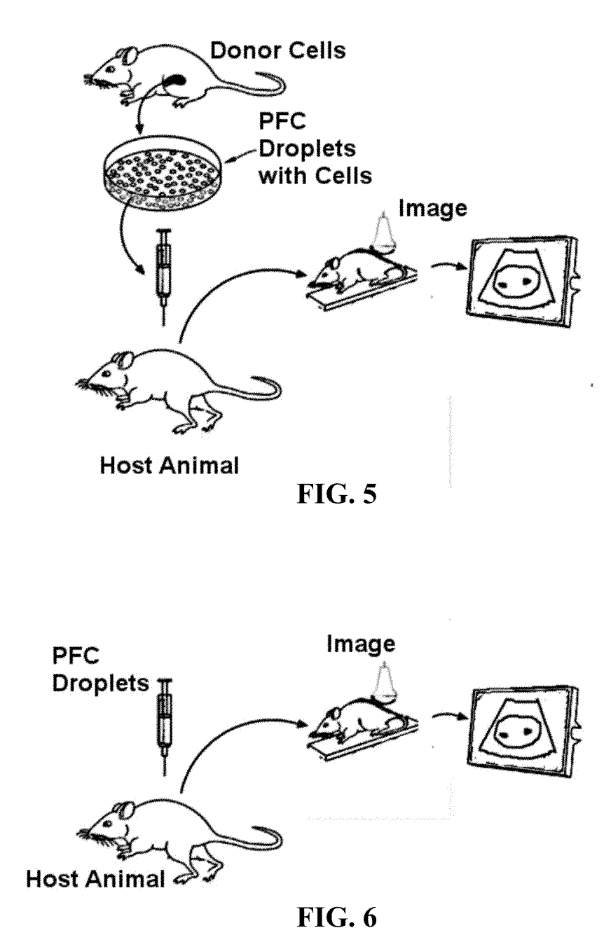

As illustrated in FIG. 5, the PFC droplets 20 can be administered to a patient by initially incubating the formed PFC droplets 20 ex vivo within donor cells from the patient or a donor. The donor cells can be isolated immune cells such as, but not limited to dendritic cells, macrophages, T-cells, and B-cells. The donor cells containing the PFC droplets 20 can be administered to the patient. In at least one example, the donor cells can be selected for use in flow cytometry if the PFC droplets have a fluorescent probe. The PFC droplets can contain antigens or other payloads that can be phagocytosed by the immune cells in a time-dependent manner. The liquid PFC core 22 can be stable within the immune cells before the PFC droplets 20 are converted to microbubbles in a controlled manner by the application of energy to provide a location of the immune cells within the patient with an ultrasound or photoacoustic imaging system.

As illustrated in FIG. 6, the PFC droplets 20 can be directly injected into a patient and ingested by phagocytic cells in vivo. The PFC droplets 20 can be used to track the movement of phagocytic cells to and from the organ or region of injection. The PFC droplets 20 can be injected into the bloodstream, lymph system, or directly into an organ or region of interest (e.g., tumor). The liquid PFC core 22 can be stable within the immune cells before the phagocytic cells are converted to microbubbles in a controlled manner by the application of energy to provide a location of the phagocytic cells within the patient with an ultrasound or photoacoustic imaging system.

### Droplet Imaging

As illustrated in FIGS. 7A-E, in an example, the PFC droplets 20 can be imaged using exogenous energy relies on the phase conversion of the liquid droplet to a gaseous microbubble, which can generate at least one indicia of the phase change including an acoustic change, a visual change, and combinations thereof. The exogenous energy can be acoustic energy supplied by an ultrasound transducer to convert the PFC droplets 20 to microbubbles and produce a detectable acoustic signal indicating the phase change of the PFC droplet 20 to microbubbles.

The characteristics of the acoustic signal can be a function of the specific PFC comprising the liquid PFC core 22. For example, PFC droplets 20 having lower boiling points can emit acoustic waves with different frequency spectra when converting to microbubbles than PFC droplets 20 having higher boiling points. In an example, different PFC droplets 20 in which the liquid PFC cores 22 comprise different PFCs can be administered to a patient. In this configuration, the unique frequency response characteristics of droplets with dissimilar core compositions can be used to determine the presence and location of different cellular targets based on the particular PFC droplets 20 that are ingested by the targeted cells or congregate at the target site.

For example, tracking of different B-cell types (e.g., B1, B2, etc.) is of significant interest to cancer and atherosclerosis immunology. B1 cells can be labeled with PFC droplets 20 containing PFC₈ (e.g., octofluorocyclobutane) while B2 cells can be labeled with PFC droplets 20 containing PFC₁₀ (e.g., decafluorocyclobutane). Upon droplet conversion with an acoustic or optical pulse, acoustic emissions with differing acoustic frequency spectra may be recorded and processed to determine the presence and location of individual B1 and B2 cells.

### Imaging System

It should be appreciated that a variety of ultrasound-related systems and methods may be utilized as part of implementing or practicing aspects of the various embodiments of the present invention.

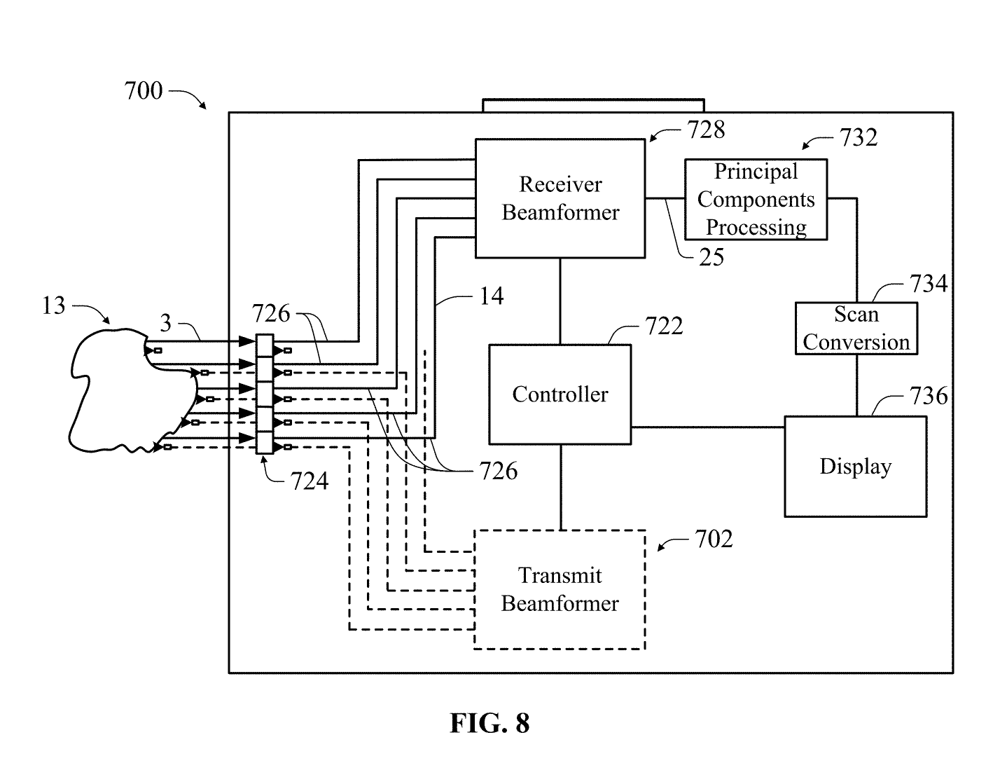

As depicted in FIG. 8, an ultrasound system 700 according to an aspect of an embodiment of the present invention that is referred to generally describe the operations of an ultrasound system to produce an image of an object 13. System 700 may optionally include a transmit beamformer 702 which may include input to it by controller 722 to send electrical instructions to array 724 as to the specifics of the ultrasonic waves to be emitted by array 724. Alternatively, system 700 may be a receive-only system, and the emitted waves may be directed to the object 13 from an external source.

In either case, echoes 3 reflected by the object 13 (and surrounding environment) are received by array 724 and converted to electrical (e.g., radio frequency (RF)) signals 726 that are input to receive beamformer 728. Controller 722 may be external of the beamformer 728, as shown, or integrated therewith. Controller 722 automatically and dynamically changes the distances at which scan lines are performed (when a transmit beamformer 702 is included) and automatically and dynamically controls the receive beamformer 728 to receive signal data for scan lines at predetermined distances. Distance/depth is typically calculated assuming a constant speed of sound in tissue (e.g., 1540 m/s or as desired or required) and then the time of flight is recorded such that the returning echoes have a known origination. The summed RF lines output by the receive beamformer 728 are input to a principal components processing module 732, which may be separate from and controlled by, or incorporated in controller 722. Principal components module 732 processes

The assembled output may be input into a scan converter module 734. The image formed within the scan converter 734 is displayed on display 736. Although FIG. 8 has been described as an ultrasound system, it is noted that transducers 724 may alternatively be transducers for converting electrical energy to forms of energy other than ultrasound and vice versa, including, but not limited to radio waves (e.g., where system 700 is configured for RADAR), visible light, infrared, ultraviolet, and/or other forms of sonic energy waves, including, but not limited to SONAR, or some other arbitrary signal of arbitrary dimensions greater than one (such as, for example, a signal that is emitted by a target).

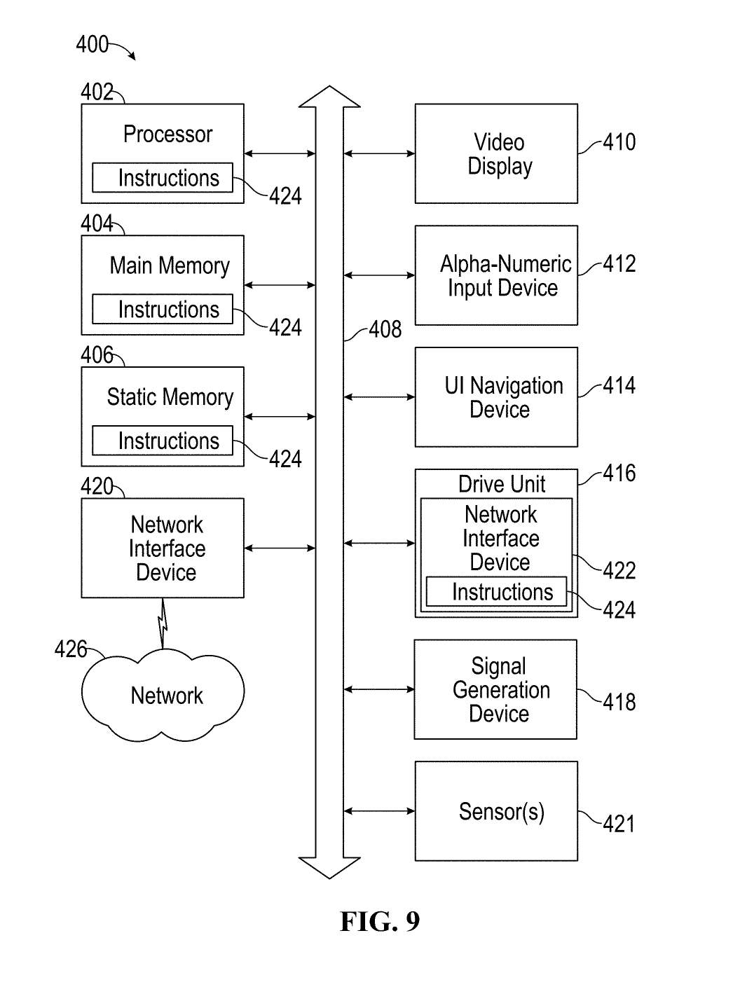

As illustrated in FIG. 9, a block diagram of an example machine 400 upon which one or more embodiments (e.g., discussed methodologies) can be implemented (e.g., run). Examples of machine 400 can include logic, one or more components, circuits (e.g., modules), or mechanisms. Circuits are tangible entities configured to perform certain operations. In an example, circuits can be arranged (e.g., internally or concerning external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine-readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or another programmable processor) that can be temporarily configured (e.g., by software) to perform the certain operations. It will be appreciated that cost and time considerations can drive the decision to implement a circuit mechanically (e.g., in dedicated and permanently configured circuitry), or in temporarily configured circuitry (e.g., configured by software).

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. The software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can operate and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine but deployed across some machines. In an example, the processor or processors can be located in a single location (e.g., within a home environment, an office environment or as a server farm), while in other examples the processors can be distributed across some locations.

### Various Notes & Examples

Example 1 is a composition for cell tracking and molecular imaging with ultrasonic energy, comprising: a perfluorocarbon (“PFC”) droplet further comprising: a liquid PFC core, a stabilizing shell material enclosing the liquid PFC core, and solid nanoparticles embedded within the liquid PFC core; wherein the solid nanoparticles act as nucleating agents promoting cavitation of the liquid PFC core to a gaseous microbubble when energy is applied to the PFC droplet.

In Example 2, the subject matter of Example 1 optionally includes that the liquid PFC core comprises a PFC comprising one of PFC₃, PFC₄, PFC₅, and PFC₆.

In Example 3, the subject matter of any one or more of Examples 1-2 optionally include wherein the liquid PFC core is saturated with oxygen.

In Example 4, the subject matter of any one or more of Examples 1-3 optionally include wherein the stabilizing shell material comprises a biocompatible lipid.

In Example 5, the subject matter of Example 4 optionally includes that the stabilizing shell material comprises a 9:1 mole ratio of 1,2-Distearoyl-sn-glycero-3-phosphocoline (“DSPC”) and 1,2-distearoyl-sn-glycero-3-phosphoethanolamine-N-[amino(polyethylene glycol)-2000 (“DSPE-PEG2000”).

In Example 6, the subject matter of any one or more of Examples 1-5 optionally include wherein the solid nanoparticles each comprise an outer surface roughened to form nanocavities on the outer surface.

In Example 7, the subject matter of Example 6 optionally includes that the nanocavities each have a radius of about 1 to about 50 nm.

In Example 8, the subject matter of any one or more of Examples 1-7 optionally include wherein the solid nanoparticles comprise at least one of mesoporous silica, gold, or iron oxide.

In Example 9, the subject matter of any one or more of Examples 1-8 optionally include wherein an outer surface of the nanoparticles are functionalized with one of a fluorine surface coating and a hydrophobic surface coating to improve the solubility of the nanoparticles within the liquid PFC core.

Example 10 is a method of forming a PFC droplet for cell tracking and molecular imaging with ultrasonic energy, comprising: providing mesoporous silica nanoparticles (“MSN”); functionalizing the MSNs with to form one of fluorinated-MSNs (“F-MSNs”) having a fluorinated outer surface or hydrophobic-MSNs (“H-MSNs”) having a hydrophobic outer surface; dispersing the functionalized MSNs in an aqueous PFC solution comprising PFCs and lipids for forming a stabilizing shell; and sonicating the aqueous PFC solution and F-MSN dispersion therein to form droplets, each droplet comprising a liquid PFC core enclosed within a stabilizing shell and containing embedded functionalized MSNs.

In Example 11, the subject matter of Example 10 optionally includes that the liquid PFC core comprises one of PFC₃, PFC₄, PFC₅, and PFC₆.

In Example 12, the subject matter of any one or more of Examples 10-11 optionally include H-Perfluorooctyltriethoxysilane (“PFOTS”).

In Example 13, the subject matter of Example 12 optionally includes washing the functionalized MSNs with an alcohol solution and a PFC wash solution before dispersion of the functionalized MSNs in the aqueous PFC solution.

In Example 14, the subject matter of any one or more of Examples 10-13 optionally include centrifuging the aqueous PFC solution containing formed droplets to separate the droplets from the aqueous PFC solution; and washing the separated droplets with a saline solution to remove excess lipids.

In Example 15, the subject matter of Example 14 optionally includes filtering the separated droplets to exclude droplets above a size threshold.

Example 16 is a method of cell tracking and molecular imaging, comprising: introducing PFC droplets into a test region such that the PFC droplets are ingested by target cells or congregate at a target site within the test region, each droplet comprising a liquid PFC core enclosed within a stabilizing shell and containing embedded functionalized MSNs; and applying energy to the test tissue in excess of a predetermined threshold causing the liquid PFC core to phase change to a gaseous microbubble thereby generating a phase change indicia comprising at least one of an auditory signal or an optical signal, wherein the functionalized MSNs act as nucleating agents encouraging cavitation of the liquid PFC core when exposed to the applied energy.

In Example 17, the subject matter of Example 16 optionally includes detecting the phase change indicia generated by the phase change of the liquid PFC core to the gaseous PFC microbubble.

In Example 18, the subject matter of Example 17 optionally includes wherein the phase change indicia from at least two directions to locate the target cells or target site within the test region.

In Example 19, the subject matter of any one or more of Examples 16-18 optionally include wherein the applied energy comprises at least one of ultrasound, other acoustic wave types, visible light, infrared, ultraviolet, radio waves, and combinations thereof.

In Example 20, the subject matter of any one or more of Examples 16-19 optionally include that the liquid PFC core comprises one of PFC₃, PFC₄, PFC₅, and PFC₆.

Each of these non-limiting examples can stand on its own, or can be combined in any permutation or combination with any one or more of the other examples.

The above-detailed description includes references to the accompanying drawings, which form a part of the detailed description. The drawings show, by way of illustration, specific embodiments in which the present subject matter can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the present subject matter should be determined with reference to the appended claims, along with the full scope of equivalents to which such claims are entitled.

What is claimed is:

1. A composition for cell tracking and molecular imaging with ultrasonic energy, comprising:
a perfluorocarbon (“PFC”) droplet further comprising:
a liquid PFC core,
a stabilizing shell material enclosing the liquid PFC core, and
solid nanoparticles embedded within the liquid PFC core;
wherein the solid nanoparticles act as nucleating agents promoting cavitation of the liquid PFC core to a gaseous microbubble when energy is applied to the PFC droplet.

2. The composition of claim 1, wherein the liquid PFC core comprises a PFC comprising one of PFC₃, PFC₄, PFC₅, and PFC₆.

3. The composition of claim 1, wherein the liquid PFC core is saturated with oxygen.

4. The composition of claim 1, wherein the stabilizing shell material comprises a biocompatible lipid.

5. The composition of claim 4, wherein the stabilizing shell material comprises a 9:1 mole ratio of 1,2-Distearoyl-sn-glycero-3-phosphocoline (“DSPC”) and 1,2-distearoyl-sn-glycero-3-phosphoethanolamine-N-[amino(polyethylene glycol)-2000 (“DSPE-PEG2000”).

6. The composition of claim 1, wherein the solid nanoparticles each comprise an outer surface roughened to form nanocavities on the outer surface.

7. The composition of claim 6, wherein the nanocavities each have a radius of about 1 to about 50 nm.

8. The composition of claim 1, wherein the solid nanoparticles comprise at least one of mesoporous silica, gold, or iron oxide.

9. The composition of claim 1, wherein an outer surface of the nanoparticles are functionalized with one of a fluorine surface coating and a hydrophobic surface coating to improve the solubility of the nanoparticles within the liquid PFC core.

10. A method of forming a PFC droplet for cell tracking and molecular imaging with ultrasonic energy, comprising:
providing mesoporous silica nanoparticles (“MSN”);
functionalizing the MSNs with to form one of fluorinated-MSNs (“F-MSNs”) having a fluorinated outer surface or hydrophobic-MSNs (“H-MSNs”) having a hydrophobic outer surface;
dispersing the functionalized MSNs in an aqueous PFC solution comprising PFCs and lipids for forming a stabilizing shell; and
sonicating the aqueous PFC solution and F-MSN dispersion therein to form droplets, each droplet comprising a liquid PFC core enclosed within a stabilizing shell and containing embedded functionalized MSNs.

11. The method of claim 10, wherein the PFCs comprise one of PFC₃, PFC₄, PFC₅, and PFC₆.

12. The method of claim 10, wherein the functionalizing step comprises reacting the MSNs with 1H, 1H, 2H, 2H-Perfluorooctyltriethoxysilane (“PFOTS”).

13. The method of claim 12, further comprising:
washing the functionalized MSNs with an alcohol solution and a PFC wash solution before dispersion of the functionalized MSNs in the aqueous PFC solution.

14. The method of claim 10, further comprising:
centrifuging the aqueous PFC solution containing formed droplets to separate the droplets from the aqueous PFC solution; and
washing the separated droplets with a saline solution to remove excess lipids.

15. The method of claim 14, further comprising:
filtering the separated droplets to exclude droplets above a size threshold.

16. A method of cell tracking and molecular imaging, comprising:
introducing PFC droplets into a test region such that the PFC droplets are ingested by target cells or congregate at a target site within the test region, each droplet comprising a liquid PFC core enclosed within a stabilizing shell and containing embedded functionalized MSNs; and
applying energy to the test tissue in excess of a predetermined threshold causing the liquid PFC core to phase change to a gaseous microbubble thereby generating a phase change indicia comprising at least one of an auditory signal or an optical signal, wherein the functionalized MSNs act as nucleating agents encouraging cavitation of the liquid PFC core when exposed to the applied energy.

17. The method of claim 16, comprising:
detecting the phase change indicia generated by the phase change of the liquid PFC core to the gaseous PFC microbubble.

18. The method of claim 17, wherein the phase change indicia from at least two directions to locate the target cells or target site within the test region.

19. The method of claim 16, wherein the applied energy comprises at least one of ultrasound, other acoustic wave types, visible light, infrared, ultraviolet, radio waves, and combinations thereof.

20. The method of claim 16, wherein the liquid PFC core comprises one of PFC₃, PFC₄, PFC₅, and PFC₆.
